(19) United States
(12) Patent Application Publication
(10) Pub. No.: US 2007/0136099 A1
(43) Pub. Date: Jun. 14, 2007

(54) Distributed medicine system

(76) Inventors: Gordon Neligh, Denver, CO (US); James O. Kemp, Littleton, CO (US)

Correspondence Address: MERCHANT & GOULD PC, P.O. BOX 2903, MINNEAPOLIS, MN, 55402-0903, US

(21) Appl. No.: 11/300,152

(22) Filed: Dec. 13, 2005

(51) Int. Cl. A61B 5/00

(52) U.S. Cl. 705/3; 600/300

(57) ABSTRACT

The embodiments presented in this document provide for distributed medicine systems and methods to treat patient with the distributed medicine system. A distributed medicine system includes a plurality of portable medical response units (PMRU), at one or more remote locations, communicatively coupled or connected to a plurality of medical treatment centers, at one or more other locations. The PMRUs can interface with one or more patients to send medical information, such as vital statistics, to one or more medical treatment centers. The medical treatment centers determine medical conditions and command the PMRUs to treat the medical conditions.

### TECHNICAL FIELD

The present invention relates, in general, to systems for monitoring and treating a medical patient in a remote location using a distributed medicine system.

### BACKGROUND

Responding to medical emergencies in remote areas or areas struck by a war or a natural disaster is extremely difficult. The remote areas often have minimal infrastructure and no medical facilities. Similarly, areas ravaged by a natural disaster often are left with a crippled or destroyed infrastructure that cannot provide rudimentary medical care. To bring medical treatment to people suffering illness in remote areas or areas destroyed by a natural disaster, new or mobile medical treatment centers are often erected. Unfortunately, the process of creating the ad hoc medical facilities takes time, and the facilities are difficult to keep open because it is generally manpower intensive to create and sustain the required medical infrastructure. In addition, some areas cannot support even a rudimentary medical hospital because of the lack of roads, electricity, water, or other support infrastructure.

It is with respect to these and other considerations that the present invention has been made.

### SUMMARY

The embodiments presented in this document provide for distributed medicine systems and methods to establish and maintain the distributed medicine systems. In embodiments, a distributed medicine system includes a plurality of portable medical response units (PMRU), also referred to as the backpack device with satellite communications (BDSC), at one or more remote locations, communicatively coupled or connected to a plurality of medical treatment centers, such as a hospital. The PMRUs can interface with one or more patients to send medical information, such as vital statistics, to one or more medical treatment centers. In further embodiments, the medical treatment centers can command the PMRUs to treat a medical condition, e.g., delivering medications or performing a medical procedure. Thus, a patient in a remote area may be assessed, observed, and treated either through directed treatment or by automatic treatment using the distributed medicine system.

A more complete appreciation of the present invention and its improvements can be obtained by reference to the accompanying drawings, which are briefly summarized below, and to the following detailed description of exemplary embodiments of the invention, and to the appended claims.

### BRIEF DESCRIPTION OF THE DRAWINGS

FIG. 1 is a schematic plan view of a distributed medicine system connecting a plurality of PMRUs with a plurality of medical treatment centers in accordance with the present disclosure.

FIG. 2 is a functional diagram illustrating a computing device operable to provide a PMRU to observe and treat a patient in accordance with the present disclosure.

FIG. 3 is a flow diagram representing an embodiment of the present disclosure for receiving medical information at medical treatment center for a patient in a remote area and treating the patient.

FIG. 4 is a flow diagram representing an embodiment of the present disclosure for connecting a PMRU to a patient, connecting the PMRU to a medical treatment center, and using the PMRU to treat the patient.

FIG. 5 is a flow diagram representing an embodiment of the present disclosure for monitoring the amount of at least one medical supply in a PMRU and requesting resupply of at least one medical supply.

FIG. 6 is a functional diagram illustrating an embodiment of the present disclosure for automatically configuring a PMRU in a remote location to connect to a medical treatment center via a predetermined communication channel.

FIG. 7 is another functional diagram illustrating a computing device operable to provide a PMRU to observe and treat a patent in accordance with the present disclosure.

FIG. 8 is a flow diagram for determining a diagnosis and possibly a treatment from two or more inputs of diagnostic data.

### DETAILED DESCRIPTION

Embodiments of the systems and methods will now be described more fully hereinafter with reference to the accompanying drawings. The described embodiments may, however, assume many different forms, and the present invention should not be construed as limited to the embodiments set forth herein. Rather, these described embodiments are provided so that the disclosure will be thorough and complete and will fully convey the scope of the possible embodiments to those skilled in the art.

The present invention generally relates to a distributed medicine system that is capable of operating in austere environments. The distributed medicine system comprises one or more PMRUs that are each configured or arranged to interface or to communicate with one or more patients. The PMRUs can gather medical information or diagnostic data, such as vital signs, and provide treatment. The treatment may be provide automatically or through some level of intervention by a technician, i.e., the treatment is fully directed by the PMRU or only partially directed by the PMRU. Thus, a technician or other personnel, without the fully trained medical abilities of a doctor, can use the PMRU to treat patients. By at least one communications interface or communication channel or connection, the PMRU can communicate with one or more medical treatment centers in one or more other locations. In embodiments, at least one medical treatment center receives medical information about a patient from the PMRU and sends a command to the PMRU to treat the patient, i.e. no medical technician or other person needs to be involved to administer a treatment after the PMRU is connected to the patient.

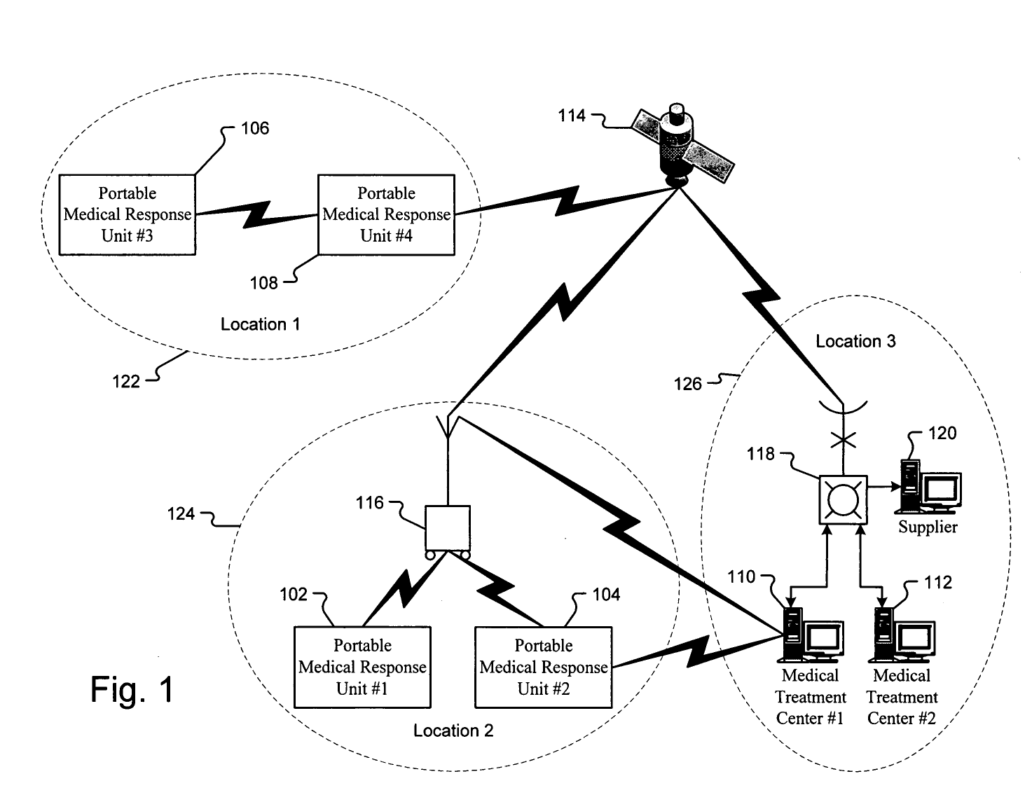

An embodiment of a distributed medicine system 100 is shown in FIG. 1. The distributed medicine system 100 comprises a plurality of PMRUs 102, 104, 106, and 108 that may be located in one or more locations, such as location 1 122 and location 2 124. The PMRUs 102, 104, 106, and 108 are self-contained units carried on or by a single person. As such, a medical technician can deploy to any area and carry all the necessary medical supplies and systems in the PMRU 102. PMRUs 102, 104, 106, and 108 gather and send medical information, such as vital statistics, to medical treatment centers, such as medical treatment center #1 110 and medical treatment centers #2 112, in another location, such as location 3 126.

The distributed medicine system 100 includes at least one PMRU that is communicatively coupled to at least one medical treatment center. In one embodiment, a PMRU, such as PMRU #4 108, transmits medical informpation directly to a satellite 114. The medical information is then relayed from the satellite 114 to a medical treatment center 110, or, in some embodiments, to a ground station 118 that is communicatively coupled to the medical treatment center 110. The satellite connection 114 can be to any orbiting satellite in any mode, frequency, or format, e.g., K band, Ka band, Ku band, bent-pipe, etc. A PMRU, such as PMRU #2 104, may also directly connect to a medical treatment center 110. In one embodiment, two or more PMRUs, such as PMRUs 102 and 104, connect to a mobile communication unit 116 that receives and manages all medical information being sent from two or more PMRUs. The mobile communication unit 116 relays the medical information to the one or more medical treatment centers 110 and 112 via a transmission link to an orbiting satellite 114. The mobile communication unit 116 may also be capable of sending the information directly to a medical treatment center 110. In still a further embodiment, a PMRU 106, may not be able to connect to either a mobile communications unit 116 or an orbiting satellite 114. The PMRU #3 106, in such situations, relays medical information to another PMRU, such as PMRU #4 108, to send the medical information to the mobile communications unit 116 or satellite 114. Throughout the description of the embodiments, communicatively coupling may mean any type of communication or electrical connection. Further, communication and electrical connections may employ any available communication technologies including, but not limited to, cellular telephone connections, WAN or LAN connections, wireless LANs, satellite communications, etc.

As explained above, the communications to the medical treatment center 110 and/or 112 may be received by a satellite tracking station 118 then transferred to the medical treatment center 110 and/or 112. The medical treatment center 110 and/or 112 may be any type of medical facility or caregiver including, but not limited to, medical clinics, private practitioners, the Center for Disease Control (CDC), National Institutes of Health (NIH), universities, pharmaceutical companies, or research institutions. The medical treatment centers 110 and 112 form a network of medical providers. Any PMRU may communicate with any medical treatment center or with more than one medical treatment center to send medical information to the medical treatment center(s) 110 and/or 112.

In further embodiments, the medical treatment center 110 and/or 112 sends commands to at least one PMRU to respond to a medical condition. The PMRU 108 receives the command and institutes a treatment response without the intervention of medical personnel at the distant location 122. Thus, the medical personnel can allow the distant medical treatment center 110 to treat a first person, connected to the PMRU 108, while the medical personnel attends to other patients. The medical treatment center 110 can send the treatment command via the same communications link used to receive the medical information or may use an alternative communication connection.

The distributed medicine system 100 provides a flexible and expandable system that can address medical needs of multiple patients in multiple locations connected to at least one PMRU with the assistance of multiple medical treatment centers. Thus, the distributed medicine system 100 is not limited to a single interface between one medical device and one distant medical treatment center. Rather, many patients may be helped by the multiple PMRUs that can communicate with the network of medical treatment centers.

Further embodiments of the distributed medicine system 100 also connects to at least one supplier 120. The supplier 120, in some situations, may be located in the same area as the PMRU or as the medical treatment center. At least one PMRU 112 monitors the amount or number of medical supplies being consumed during the treatment of one or more patients. At some threshold, e.g. one gauze pad out of 50 remains in the PMRU 112, the PMRU 112 sends a resupply request to the supplier via the satellite connection 114. The supplier 120 responds to the resupply request by sending addition supplies, e.g., 50 more gauze pads.

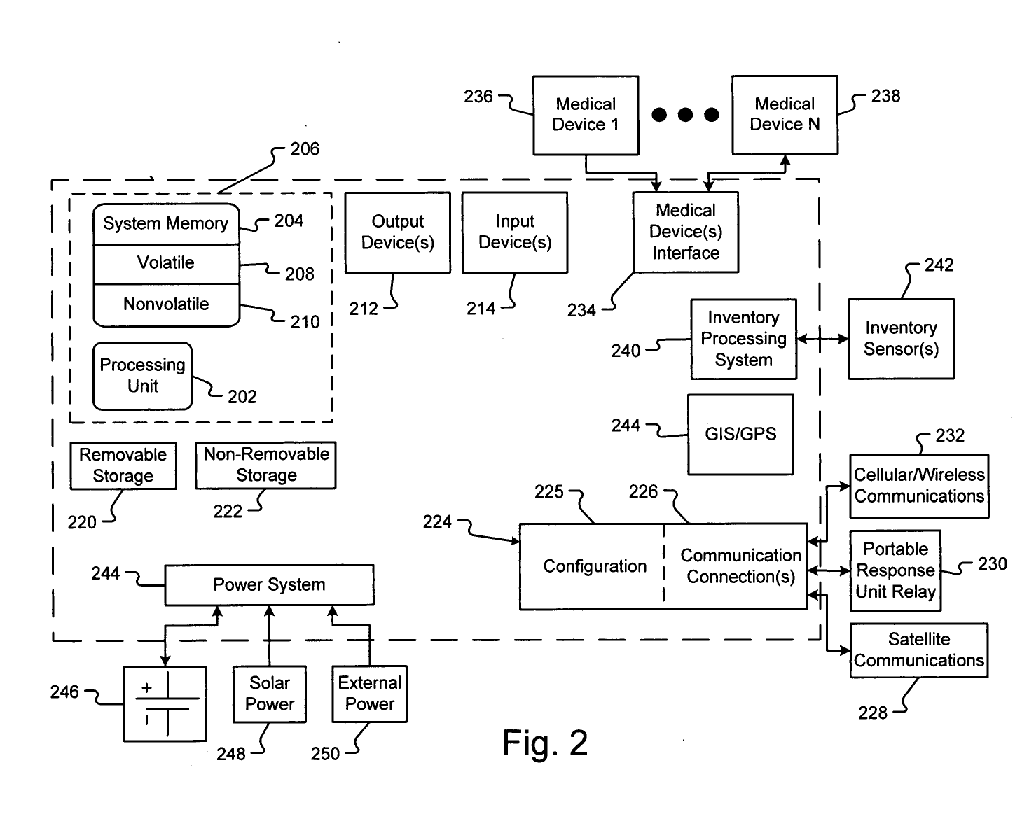

An exemplary PMRU 200, in which the invention may be implemented, is illustrated in FIG. 2. The PMRU 200 is only one example of a suitable PMRU and is not intended to suggest any limitation as to the scope of use or functionality of the invention. Other well known computing systems, environments, and/or configurations that may be suitable for use with the invention include, but are not limited to, any portable version of personal computers, server computers, hand-held or laptop devices, multiprocessor systems, microprocessor-based systems, programmable consumer electronics, network PCs, minicomputers, mainframe computers, distributed computing environments that include any of the above systems or devices, and the like.

With reference to FIG. 2, an exemplary PMRU 200 includes a computing device 206. In its most basic configuration, computing device 206 typically includes at least one processing unit 202 and memory 204. Depending on the exact configuration and type of computing device 206, memory 204 may be volatile 208 (such as RAM), non-volatile 210 (such as ROM, flash memory, etc.), or some combination of the two. The most basic configuration of the computing device 200 is illustrated in FIG. 2 by dashed line 206. Additionally, PMRU 200 may also have additional features or functionality. For example, PMRU 200 may also include additional storage (removable and/or non-removable) including, but not limited to, magnetic or optical disks or tape. Such additional storage is illustrated in FIG. 2 by removable storage 220 and non-removable storage 222. Computer storage media includes volatile and nonvolatile, removable and non-removable media implemented in any method or technology for storage of information, such as computer readable instructions, data structures, program modules, or other data. Memory 204, removable storage 220, and non-removable storage 222 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by PMRU 200. Any such computer storage media may be part of PMRU 200.

PMRU 200 may also contain communication interface 224 that allows the PMRU 200 to communicate with other devices, such as satellite communications equipment 228, PMRU relay unit 230, or cellular/wireless communications equipment 232. Communication interface 224 is an example of communication media. Communication media typically embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media.

In embodiments, the communication interface 224 comprise a configuration system 225 and communication connection(s) 226. Configuration system 225 is arranged to receive configuration information from a permanent communication connection and configure communication connections 226. For instance, the configuration information may request the configuration system 225 to configure a communication connection 226 to communicate through satellite communications system 228 to an orbiting satellite, such as satellite 114 (FIG. 1). Configuration information may comprise any information to configure communication connection 226 to communicate via any type of system, communication media, or any format.

PMRU 200 may also have input device(s) 214 such as keyboard, mouse, pen, voice input device, touch input device, medical device, etc. Output device(s) 212, such as medical devices, a display, speakers, printer, etc. may also be included. All these devices are well know in the art and need not be discussed at length here.

Computing device 200 typically includes at least some form of computer readable media. Computer readable media can be any available media that can be accessed by processing unit 202. By way of example, and not limitation, computer readable media may comprise computer storage media and communication media. Combinations of the any of the above should also be included within the scope of computer readable media.

The computer device 200 may operate in a networked environment using logical connections to one or more remote computers, such as a computer at the medical treatment center 110 (FIG. 1) in distant location 3 126 (FIG. 1). The remote computer may be a personal computer, a server computer system, a router, a network PC, a peer device, another computer system, or other common network node, and typically may include many or all of the elements described above relative to the PMRU 200. The logical connections between the PMRU 200 and the remote computer may include a local area network (LAN), a wide area network (WAN), or other networks, such as a satellite network connection. Such networking environments are commonplace in offices, enterprise-wide computer networks, intranets, and the Internet.

When used in a LAN networking environment, the PMRU 200 is connected to the LAN through a network interface or adapter. When used in a WAN networking environment, the computer device 200 typically includes a modem or other means for establishing communications over the WAN, such as the Internet. The modem, which may be internal or external, may be connected to the computer processor 202 via the communication connections 224, or other appropriate mechanism. In a networked environment, program modules or portions thereof may be stored in the remote memory storage device. By way of example, and not limitation, remote application programs may reside on memory device connected to the remote computer system. It will be appreciated that the network connections explained are exemplary and other means of establishing a communications link between the computers may be used.

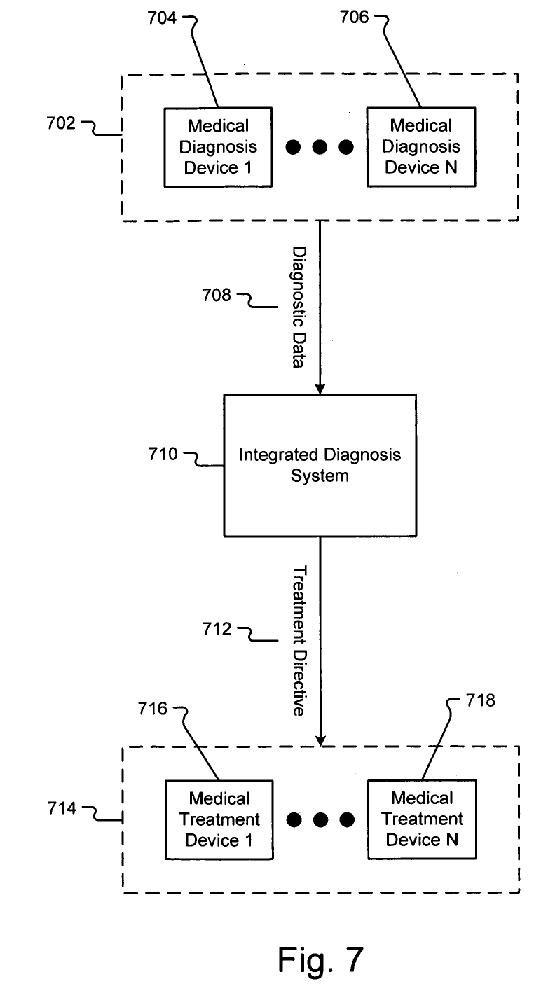

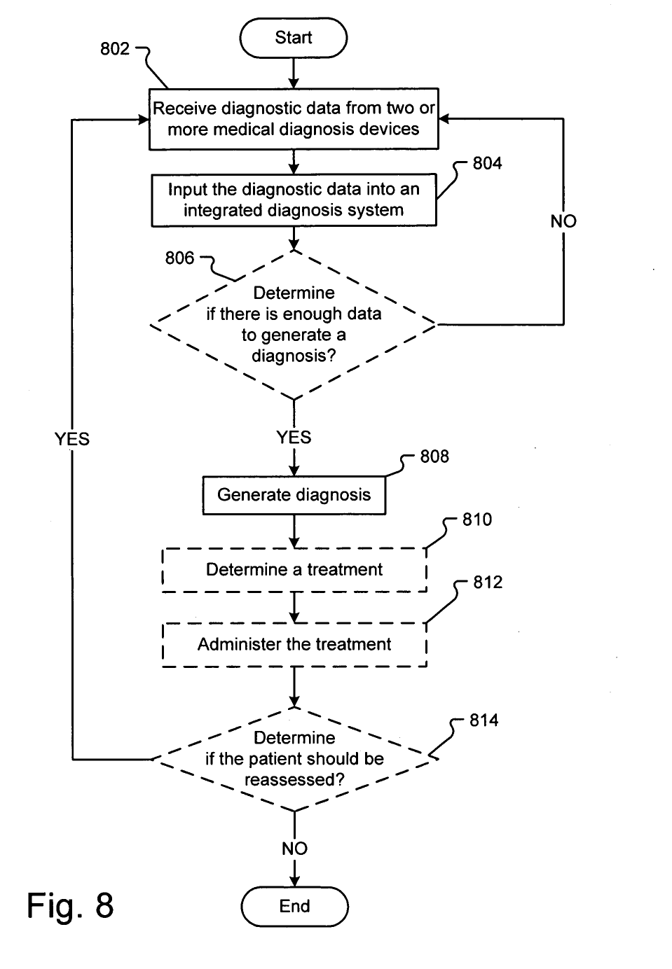

The PMRU 200 also comprises a medical device interface 234, which interfaces, connects, or communicates with one or more medical devices 236 through 238. The medical devices 236 through 238 may include, but are not limited to, electrocardiogram sensors, sonograms, blood chemistry sensors, pulse oximeters, intravenous fluid delivery systems, medication delivery systems, thermometers, gas delivery systems, or other medical systems known in the art. The medical devices 236 through 238 can provide medical information to the PMRU for transmission to a remote medical treatment center or may receive commands from one or more remote medical treatment centers to deliver a treatment to a connected patient. Medical information comprises any information regarding the patient, such as name, height, weight, heart rate, blood pressure, etc. Vital statistics, such as heart rate, blood pressure, or other critical medical data, are part of the medical information that is received from the medical devices 236 through 238 and send to the remote medical treatment centers. In embodiments, observations from a technician may also be input into the PMRU through a man/machine interface. In situations where the PMRU cannot connect to a remote medical treatment center, the PMRU is capable of operating in stand alone mode wherein the PMRU diagnosis the patient automatically, as explained in conjunction with FIGS. 7 and 8.

In embodiments, the PMRU 200 also includes an inventory processing system 240 that receives signals from at least one inventory sensors 242. The inventory sensors 242 include any electrical or mechanical system capable of measuring the volume or quantity of a medical supply in or on the PMRU 200. For example, amount of gas or fluids in a fluid or gas delivery system may be measured with a pressure sensor. In another example, an amount of physical supplies, such as the number of gauze pads, may be measured with a bar code scanner or similar scanning system. The inventory processing system 240 receives the inputs from inventory sensors 242. The inventory processing system 240 determines if the quantity or volume of the medical supply reaches a threshold that requires a resupply request to be sent. In embodiments, the inventory processing system 240 comprises a processing unit that can compare the amount or number of units of a medical supply with a predetermined threshold. If the threshold is reached or crossed, the inventory processing unit 240 may send a resupply request to the processing unit 202 for relay to the communication interface 224.

In further embodiments, the PMRU 200 also comprises a geographic information system (GIS) and/or a geographic positioning system (GPS). The GIS/GPS unit 244 is any system, as is known in the art, for receiving signals from a GPS satellite and translating the signals into a geographic position. To provide a location of the PMRU 200 for resupply, the GIS/GPS unit 244 can determine a location for the PMRU 200 and send the location to the processing unit 202. The processing unit 202 can include the location in the resupply request.

A great advantage of the PMRU 200 is its ability to operate without any outside infrastructure or support systems. The independence of the PMRU 200 is possible because of the power system 244. The power system 244 converts, generates, or receives power from one or more sources. In austere environments, the power system 244 can select and operate from a connected battery 246. The battery 246 is any battery for a mobile device with sufficient power capabilities and having lightweight, such as a battery for a laptop computer. In addition, the power system 244 may be able to receive power from a connected solar cell array and system 248 carried with or on the PMRU 200. The solar cell array and system 248 can be any solar cell system known in the art that can be carried with the PMRU 200. In embodiments, the power system 200, when possible, can connect to an external power source 250, such as a power grid or network or generator. Regardless of power source, the power system 244 can convert the incoming power into a usable voltage and/or current and provide the power to the PMRU 200 or use the power to recharge the battery 246.

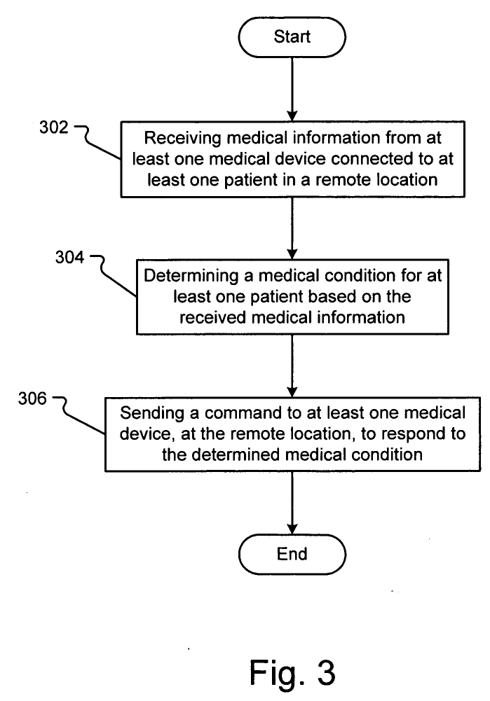

A method 300 for responding to a medical emergency monitored by a PMRU, such as PMRU 102 (FIG. 1), is shown in FIG. 3. Receive operation 302 receives medical information at a medical treatment center, such as medical treatment center 110 (FIG. 1), from one or more medical devices, such as medical device 236 (FIG. 2), connected to a patient at a remote location, such as location 2 124 (FIG. 1). The medical information is sent to the medical treatment center from through a PMRU communicatively coupled to the medical device. The PMRU transmits the medical information over one or more communication's pathways, such as satellite communication pathway 114 (FIG. 1). In embodiments, the medical information provides vital statistics for the patient that reveals or indicates one or more medical conditions requiring treatment.

Determine operation 304 determines at least one medical condition suffered by the patient in the remote location based on the medical information received from the one or more medical devices. In determining the medical condition, a doctor or other caregiver, in embodiments, reviews the incoming medical information. From the medical information, the caregiver diagnoses a condition suffered by the patient.

Send operation 306 sends a command to the PMRU, and the command is relayed to another medical device connected to the patient at the remote location to treat the patient's diagnosed condition. In one embodiment, the caregiver enters a command into a computing device at the medical treatment center and transmits the command to the PMRU. Upon receiving the command, the PMRU relays the command to the appropriate medical device to deliver treatment.

For example, a patient at a remote location may be connected to an electrocardiogram (ECG) and a defibrillator. The PMRU collects ECG telemetry from the ECG and sending the ECG information to a remote medical treatment center. A doctor at the medical treatment center reviews the ECG information as it is received. The doctor may then determine that the patient is entering or in cardiac arrest. The doctor responds by entering a command into a computer system at the medical treatment center to activate the defibrillator. The PMRU receives the command and relays the command to the defibrillator, which automatically configures to the appropriate setting according to the command. The defibrillator delivers a shock, the treatment to restart a heart in cardiac arrest, to the patient in response to the command. As can be deduced from this example, the distributed medicine system can provide medical information from a PMRU and use the PMRU to treat a patient without intervention by a medical technician or other person.

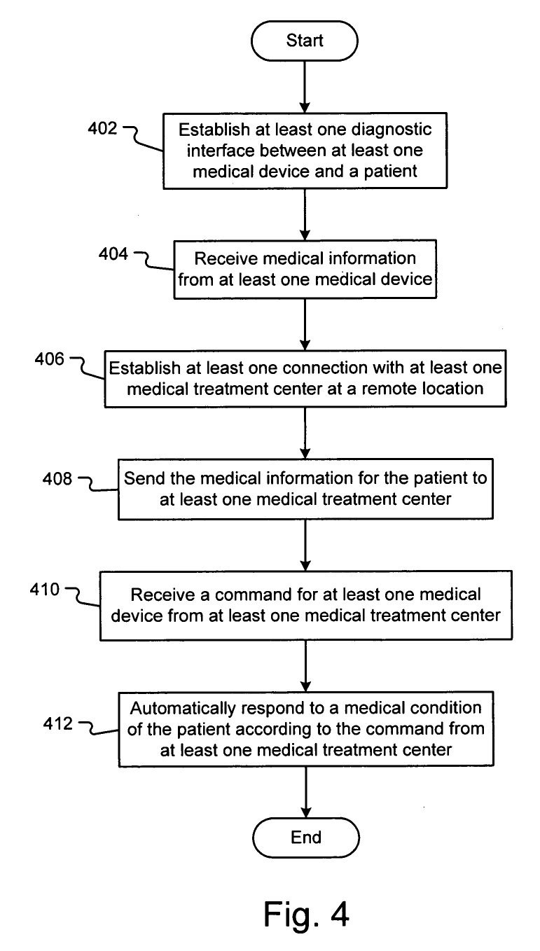

Another embodiment of a method 400 for treating a patient with a PMRU, such as PMRU 101 (FIG. 1), in a remote location, such as location 124 (FIG. 1), is illustrated in FIG. 4. Establish operation 402 establishes a diagnostic interface between the patient and at least one medical device, such as medical device 236 (FIG. 2). In embodiments, two or more medical devices in the PMRU are connected to the patient. For example, a pulse oximeter is placed on the patient's finger or an intravenous fluids delivery system is inserted into a patient's vein. The diagnostic interface(s) between the medical device and the PMRU allows the PMRU to collect medical information and/or respond to a medical condition. Receive operation 404 receives the medical information from the medical device. In one embodiment, the PMRU receives medical information, e.g., vital statistics, from the one or more medical devices. The medical information can be collected and formatted for transmission to a remote medical treatment center, such as medical treatment center 110 (FIG. 1).

Establish operation 406 establishes a connection with a medical treatment center in a remote location, such as location 126 (FIG. 1). In one embodiment, the PMRU connects to the remote medical treatment center through a satellite connection, such as satellite connection 114 (FIG. 1). In other embodiments, the PMRU connects directly to the medical treatment center or relays message through another PMRU using a wireless or cellular communications connection. A mobile communications unit, such as mobile communications unit 116 (FIG. 1), in still other embodiments, connects with the PMRUs and relays medical information to the medical treatment centers.

Once a connection is made, send operation 408 sends medical information for one or more patients to the remote medical treatment center. In one embodiment, the PMRU multiplexes or formats medical information for two or more patients into a single transmission signal and sends the single transmission signal to the remote medical treatment center. After sending the medical information, receive command 410 receives a command at the PMRU to send or relay to one or more medical devices. The command may instruct or direct one or more medical devices to reconfigure the device, reformat the medical information, or perform a medical treatment. Then, response operation 412 responds to the command for medical treatment. For instance, a command may be sent to the intravenous fluid delivery system connected to the patient to administer a drug, such as a pain reliever. Thus, the PMRU can address medical conditions without the action of a medical technician at the remote location.

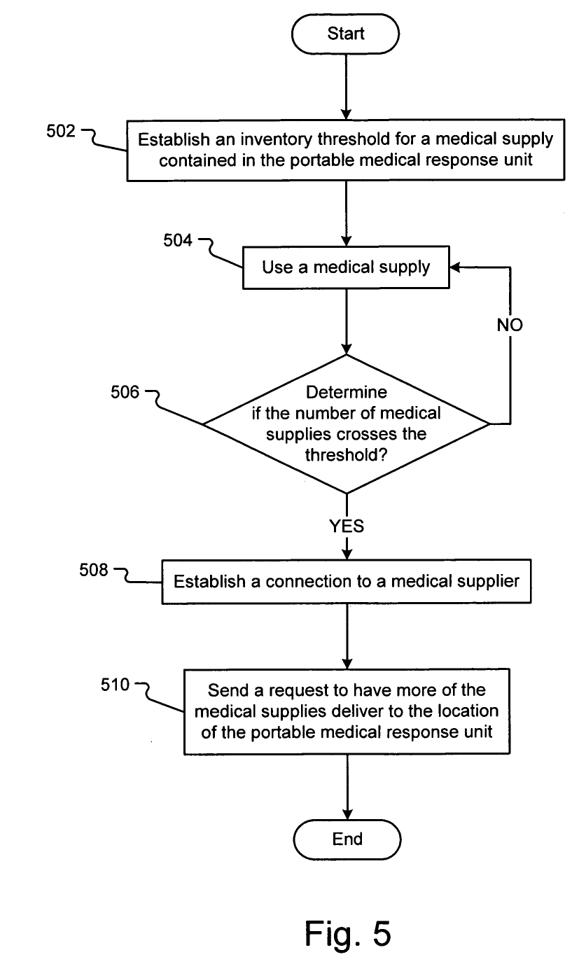

A method 500 for automatically requesting medical supplies for a PMRU, such as PMRU 102 (FIG. 1), is shown in FIG. 5. Establish operation 502 establishes an inventory threshold for at least one medical supply contained within a PMRU. In one embodiment, the threshold is predetermined by input. For example, an inventory control manager may determine that the inventory for a medical supply cannot be less than two items without requesting a resupply. The chosen number is then assigned as the threshold. In another embodiment, the threshold is determined. For example, the threshold may be designated as 20% of all supplies in the PMRU. Thus, if the PMRU holds 10 items of a certain medical supply then the request for resupply threshold would be two. When the number of medical supplies reaches two, a request for resupply is needed. In still other embodiments, the threshold may be based on how fast the supplies are being used. For example, if there are 10 supplies and one supply per hour is being consumed, the threshold may be set such that resupply happens before exhausting he supplies. For instance, if resupply takes two hours, the threshold may be set at two supplies.

Use operation 504 uses a medical supply. In embodiments, a medical technician uses the medical supply to treat a patient at a remote location. Determine operation 506 determines if the current inventory, after the technician uses a supply, reaches or is less than the threshold. In embodiments, an inventory processing system, such as inventory processing system 240 (FIG. 2), determines the number of supplies that have been used and subtracts that number from the original inventory. If the derived inventory is not less than the threshold, the method flows NO back to use operation 504. However, if the derived inventory is equal to or less than the inventory threshold, the method flows YES to establish operation 508.

Establish operation 508 establishes a connection with a medical supplier, such as supplier 120 (FIG. 1), that can resupply the PMRU. In one embodiment, the PMRU connects to the supplier through a satellite connection. Send operation 510 sends the resupply request from the PMRU to the supplier via the communication connection. The resupply request alerts the supplier that more medical supplies are needed at the location of the PMRU. In embodiments, a set of GPS coordinates, from a GPS unit, such as GPS unit 244 (FIG. 2), in the PMRU, are also transmitted with the request such that the supplier may be able to locate the PMRU for later resupply. In still further embodiments, the type of supply needed is also sent to the supplier to allow the supplier to resupply the correct medical supply. For instance, if the PMRU requires more gauze, the resupply request may identify gauze as the required supplied by, for example, sending an inventory number or identifier with the resupply request.

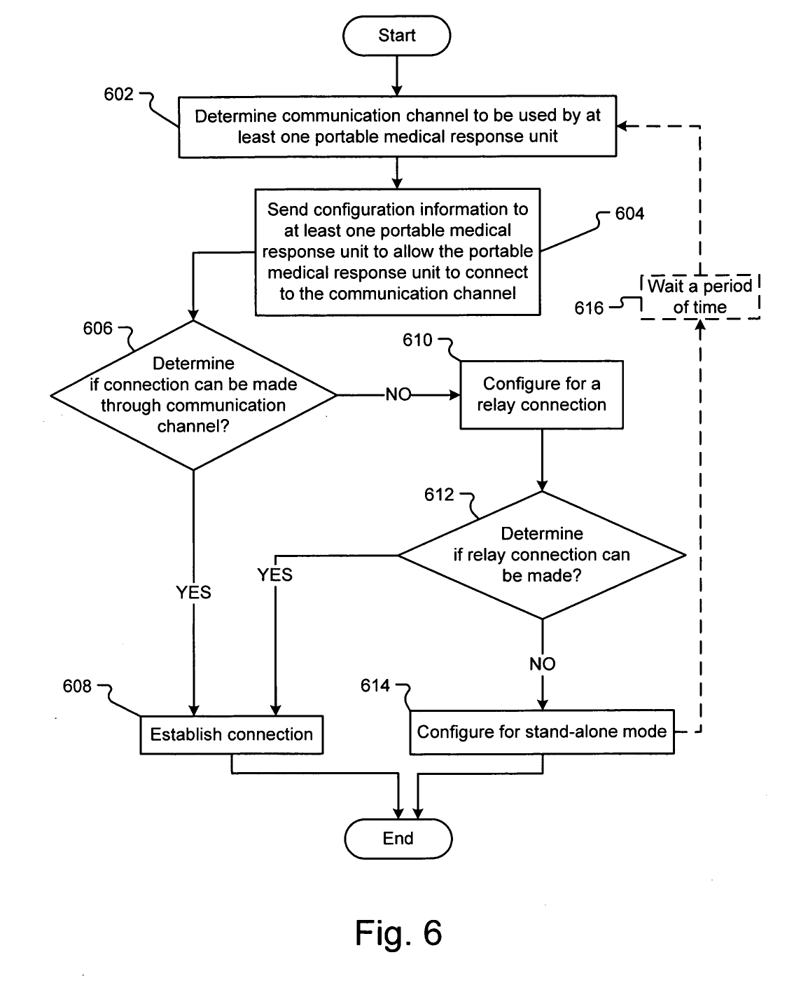

A method 600 for automatically configuring the PMRU, such as PMRU 102, is shown in FIG. 6. Determine operation 602 determines what type of communication channel to use and identifies the communication channel that will be used. For example, if the only possible connection from the PMRU to the remote medical treatment center, such as medical treatment center 112 (FIG. 1), is through a satellite connection, such as satellite connection 114 (FIG. 1), then a satellite connection is determined to be used. In addition, the particular satellite, frequency, satellite orbital information, or other information required to form the satellite connection is also determined. For example, the determined satellite connection may be through the ARABSAT-3A satellite using a Ku-Band transmission to a transponder having a specific identifier.

Send operation 604 sends the required configuration information to a PMRU. In embodiments, the PMRU is permanently connected to a configuration system via a communication connection. For example, the PMRU is always able to receive communications over a certain frequency, narrowband, satellite transmission from one or more satellites. The connection allows the PMRU to be configured for a broadband connection through another satellite. As such, the configuration information is sent to the PMRU via the narrowband connection. The PMRU receives the configuration information at a configuration system, such as configuration system 225 (FIG. 2), and configures the communications system, such as communication connections 226 (FIG. 2), according to the configuration information.

Determine operation 606 determines if the connection can be made. The PMRU, after configuring, tries to establish the connection to the communications interface. For example, the PMRU sends a sync command to the satellite to establish a connection. If the satellite connection is established, the PMRU can then send a handshake signal to a remote medical treatment center to establish a communication connection with a remote medical treatment center. If the communication connection is determined possible, e.g., the sync was received and processed by the satellite, then the method flows YES to establish operation 608 to establish the connection. However, if the communication connection is determined to not be possible, the method flows NO to configure operation 610.

Configure operation 610 configures the PMRU to attempt a relay connection to another PMRU. A relay connection may be a connection through another PMRU or a connection through a mobile communication unit, such as mobile communication unit 116 (FIG. 1). The relay connection allows the PMRU to connect to a remote medical treatment center without connecting to a satellite or directly to the medical treatment center. For example, if the PMRU is inside a building, the PMRU may be unable to connect directly to a satellite. In this situation, the PMRU connects with another PMRU outside the building, and the other PMRU relays the medical information or the commands between the PMRU inside the building and the satellite. In another embodiment, a mobile communications unit relays the medical information and the commands between the PMRU and the remote medical treatment center.

Determine operation 612 determines if a relay connection can be made. If the connections can be made, the method flows YES to establish operation 608 to establish the connection. If the connections cannot be made, the method flows NO to configure operation 614. Configure operation 614 configures the PMRU to operate in stand-alone mode with no connection to a remote medical treatment center. Thus, the PMRU can still treat patients without the connection. In a further embodiment, if a PMRU is configured in stand-alone mode, the method may flow to wait operation 616. Wait operation 616 waits a period of time, such as 15 minutes, then the method begins again at determine operation 602. In this manner, the PMRU may lose and reestablish a communication connection automatically.

Another embodiment of a PMRU is shown in FIG. 7. In the exemplary embodiment, two or more medical diagnosis devices 702 are connected or communicatively coupled to an integrated diagnosis system 710. In addition, the integrated diagnosis system 710 is also connected to two or more medical treatment devices 714. The medical diagnosis devices 702 may be the same or different physical instrument or device from the medical treatment devices 714, but for more simple explanation, the medical diagnosis devices 702 and the medical treatment devices 714 appear separate. In addition, the medical diagnosis devices 702 and the medical treatment devices 714 may be the same or similar to any of the medical devices 236 through 238 described in conjunction with FIG. 2. For example, the same device can measure and provide heart rate information and also provide defibrillation if the patient is in cardiac arrest. Each medical diagnosis device 704 to 706 provides one or more types of diagnostic data 708 to the integrated diagnosis system 710. Likewise, each medical treatment device 716 to 718 provides one or more medical treatments to the patient.

The integrated diagnosis system 710 provides a system for interpreting the two or more diagnostic data inputs 708. Rather than just determining a problem using a single type of diagnostic data, the integrated diagnosis system 710 integrates the various diagnostic data 708 into a single diagnosis. As such, while one input of diagnostic data may yield a particular diagnosis, e.g., a high body temperature is the result of heat stroke, several inputs of diagnostic data may yield a very different diagnosis, e.g., a high body temperature and a high white blood cell count is the result of an infection. Thus, the accuracy of the diagnosis from the integrated diagnosis system 710 is related to the type and number of diagnostic data inputs 708. The integrated diagnosis system 710 can also generate a treatment directive 712 based on the diagnosis, e.g., administer antibiotics for the infection. The treatment directive 712 can be an automatic command to a medical treatment device 714, such as commanding a defibrillator to administer a shock to a patient in cardiac arrest, or may be a directive to a technician, e.g., inject the patient with 10 cc's of a broad spectrum antibiotic.

In the exemplary embodiment, there are three types medical software for the PMRU. The first type of software is “instant-on” software, resident in the integrated diagnosis system 710 for immediate medical emergencies. The instant-on software is limited in size, memory usage, and possibly stored on a PROM, ROM, or RAM to be available nearly simultaneously, e.g., several seconds, with the turning on of the PMRU. In one embodiment, the instant-on software includes device drivers and instructions for personal protection, cardiac monitoring, cardiac rhythm interpretation, defibrillation, emergency respiratory assistance, spinal stabilization, and bleeding control. The instant-on software needs to be available within the few seconds of turning on the PMRU for quick response to medical emergencies. The availability and performance of this software must be as “fail safe” as possible because the first few seconds of medical intervention may be life saving.

The second type of software is stored on a more permanent storage system, e.g., a hard drive, of the PMRU. This second type of software may include other device drivers for medical diagnostic and treatment devices and basic software to provide information about the most basic medical diagnoses and treatments for less emergent medical situations and operates if the satellite communication system is unavailable. In the exemplary embodiment, the second type of software includes medical information for a medical technician, such as directions to splint fractures and sprains, move patients with spinal injuries, treat severe burns, administer cardiac medications for less severe cardiac arrhythmias, treat severe anaphylactic allergic reactions, suture skin lacerations, provide ongoing respiratory support, intravenous fluid, and electrolyte replacement, treat a patient with pneumothorax, and directions for many other medical treatments. In further embodiments, the second type of software provides a system to interface with the medical technician and question the medical technician for diagnostic data, such as determining the cause of respiratory insufficiency, diagnosing fractures, etc. In one embodiment, the second type pf software can be upgraded or updated through the satellite connection, when available, at periodic intervals.

The third type of software is available with the satellite uplink to the remote treatment center(s). Once a satellite connection is established, the third type of software may either connect the medical technician, in the field, with a physician at a treatment center or with a large database of diagnostic and treatment information. The physician can send computerized information as explained in conjunction with FIG. 1, illustrations of conditions (such as circulatory or nerve pathway diagrams), or photographs of procedures. The third type of software may also provide patient information in various languages for medical technicians in other countries.

In a further embodiment, the integrated diagnosis system 710 determines that more diagnostic data is required for an accurate diagnosis. For example, the integrated diagnosis system 710 may yield three (3) different diagnoses from the present diagnostic data 708. If one more type of diagnostic data is input, the integrated diagnosis system 710 could determine which diagnosis is correct. The integrated diagnosis system 710 then either automatically acquires or requests the additional diagnostic data 708.

A method for automatically determining a diagnosis and possibly a treatment from two or more inputs of diagnostic data is shown in FIG. 8. Receive operation 802 receives diagnostic data, such as diagnostic data 708 (FIG. 7), from two or more medical diagnosis devices, such as medical diagnosis devices 702 (FIG. 7). Input operation 804 inputs the diagnostic data into an integrated diagnosis system, such as integrated diagnosis system 710 (FIG. 7).

Optional determine operation 806 determines if there is enough data to generate a diagnosis. For example, the integrated diagnosis system may conclude that one or more of twelve different ailments or disorders may be causing the present patient condition. As such, the integrated diagnosis system may then determine that one or more other types of diagnostic data input can differentiate which of the twelve ailments or disorders are causing the patient's symptoms. If there is not enough data, the process flows NO back to receive operation 802 to receive additional diagnostic data. In embodiments, one or more other medical diagnosis devices are interfaced for addition data, and, in some embodiments, the integrated diagnosis system instructs a technician to connect or reconfigure one or more medical diagnosis devices. In still other embodiments, the integrated diagnosis system requests an observation from the technician, e.g., “What color is the patient's skin” (A) Yellow, (B) Blue, (C) White, or (D) Orange.”

If there is enough diagnostic data, generate operation 808 generates a diagnosis. In further embodiments, optional determine operation 810 determines a treatment for the generated diagnosis. For example, if the diagnosis is dehydration, the treatment may be the administration of intravenous liquids. In still further embodiments, optional administer operation 812 administers the determined treatment. For example, instructions for placement of an IV are given to a technician. The appropriate type and amount of fluids are presented to the technician to place on the IV. In other embodiments, the integrated diagnosis system automatically treats the patient. For example, the integrated diagnosis system automatically delivers a shock to the patient to respond to a diagnosed cardiac arrest.

Finally, optional determine operation 814 determines if the patient should be reassessed. Each medical treatment may include several serial or parallel diagnoses with several serial or parallel treatments. Each patient may suffer from one or more ailments wherein a first ailment is treated, the patient is reassessed, and a second or more treatments follow after the reassessment. In addition, many ailments require constant or additional diagnosis or monitoring and treatment. For example, a hypothermic patient may need constant monitoring while the patient's body temperature is returned to normal because hypothermia may cause other medical problems, such as cardiac arrest. If reassessment is required, the process flows YES to receive operation 802. One or more of the previous steps may then be repeated. In other embodiments, the process flows YES to another operation, such as the determine operation 806. If there is no need to reassess the patient, i.e., the patient is healed, the process flows NO to end the process.

Although the present disclosure has been described in language specific to structural features, methodological acts, and computer readable media containing such acts, it is to be understood that the appended claims are not necessarily limited to the specific structure, acts, or media described. One skilled in the art will recognize other embodiments or improvements that are within the scope and spirit of the present disclosure. Therefore, the specific structure, acts, or media are disclosed as exemplary embodiments. The invention is defined by the appended claims.

What is claimed is:

1. A distributed medicine system for treating a patient in a remote location, comprising:
two or more portable medical response units at a first location, wherein each portable medical response unit is in communication with at least one patient;
two or more medical treatment centers at a second location, wherein each medical treatment center receives medical information about at least one patient from at least one portable medical response unit, at least one medical treatment center sends commands to at least one portable medical response unit to respond to a medical condition of at least one patient; and
at least one communications interface that communicatively couples at least one portable medical response units at the first location to at least one medical treatment center at the second location.

2. A distributed medicine system defined in claim 1, further comprising:
a second portable medical response unit at a third location communicatively coupled to at least one medical treatment center; and
a second communications interface that communicatively couples the second portable medical response units at the third location to at least one medical treatment center.

3. A distributed medicine system defined in claim 1, wherein the communications interface is a satellite connection, a wireless connection, or a cellular connection.

4. A distributed medicine system defined in claim 1, further comprising:
a mobile communications unit that is communicatively coupled to two or portable medical response unit, the mobile communications unit is also communicatively coupled to at least one medical treatment center; and
wherein the mobile communications unit formats the medical information from the two or more portable medical response units into a data signal and sends the data signal via the communications interface.

5. A distributed medicine system defined in claim 1, wherein at least one portable medical response unit relays medical information from one or more other portable medical response units to at least one medical treatment center via the communications interface.

6. A distributed medicine system defined in claim 1, further comprising:
a supplier at a fourth location, wherein the supplier receives medical supply information from at least one portable medical response unit.

7. A distributed medicine system defined in claim 1, wherein the portable medical response unit comprises:
a processing unit;
a medical device interface communicatively coupled to the processing unit and at least one medical device, wherein the medical device interface receives medical information from at least one medical device and sends the medical information to the processing unit; and
a communications system communicatively coupled to the processing unit, the communications system configured to receive medical information from the processing unit and send the information to at least one medical treatment center via one or more communication connections.

8. A distributed medicine system defined in claim 7, wherein the portable medical response unit further comprises:
at least one inventory sensor that measures an amount of medical devices being consumed;
an inventory processing system communicatively coupled to at least one inventory sensor and the processing unit, the inventory processing system receives the measured amount of medical devices and sends a request for resupply to the processing unit for transmission to a supplier.

9. A distributed medicine system defined in claim 7,.wherein the portable medical response unit further comprises:
a power system electrically connected and configured to obtain power from one or more power sources such that the portable medical response unit need not require connection to an external power source.

10. A distributed medicine system defined in claim 7, wherein the portable medical response unit further comprises:
a communication configuration unit communicatively coupled to at least one communications interface and electrically connected to the communications system such that the communication configuration unit receives configuration information from the communications interface and configures the communications system in response to the configuration information.

11. A method executable by a distributed medicine system for treating a patient in a remote location, comprising:
receiving a medical information for at least one patient connected to at least one portable medical response unit in the remote location;
determining a medical condition for at least one patient based on the medical information; and
sending a command to at least one portable medical response unit to respond to the determined medical condition.

12. A method for treating a patient defined in claim 11, wherein medical information includes vital statistics.

13. A method for treating a patient defined in claim 11, wherein the command instructs a medical device connected to the portable medical response unit to treat the determined medical condition.

14. A method for treating a patient defined in claim 13, wherein the medical device administers a medication.

15. A method for treating a patient defined in claim 13, wherein the medical device performs a medical procedure.

16. A method executable by a portable medical response unit for automatically requesting resupply of a portable medical response unit, comprising:
establishing an inventory threshold for a medical supply in the portable medical response unit;
using a medical supply;
determining if a number of medical supplies used reaches the threshold;
if the number of medical supplies used reaches the threshold, automatically sending a request for resupply to a supplier.

17. A method executable by a portable medical response unit for automatically configuring a communication connection with a portable medical response unit, comprising:
receiving configuration information to connect the portable medical response unit to a predetermined communications channel;
determining if a connection through the communication channel can be made;
if the connection can be made, automatically establishing the connection.

18. A method for automatically configuring a communication connection defined in claim 17, further comprising:
if the connection cannot be made, automatically configuring for a relay connection;
determining if the relay connection can be made; and
if the relay connection can be made, automatically establishing the relay connection.

19. A method for automatically configuring a communication connection defined in claim 18, further comprising:
if the relay connection cannot be made, configuring the portable medical response unit for stand-alone mode.

20. A method for automatically configuring a communication connection defined in claim 18, wherein the relay connection is through another portable medical response unit or a mobile communication unit.

21. A method executable by a portable medical response unit for determining diagnosis and administering a treatment to a patient, comprising:
(a) receiving diagnostic data from two or more diagnostic medical devices;
(b) determining a diagnosis from the diagnostic data from the two or more diagnostic medical devices;
(c) determining a treatment to respond to the determined diagnosis; and
(d) administering the treatment.

22. A method defined in claim 21, wherein determining a diagnosis further comprises:
determining if there is enough diagnostic data to determine a diagnosis; and
if there is not enough diagnostic data, requesting additional diagnostic data.

23. A method defined in claim 21, further comprising:
determining if the patient should be reassessed: and
if the patient should be reassessed, repeating steps (a), (b), (c), or (d) of claim 21.

24. A portable medical response unit for determining a diagnosis and administering a treatment to a patient, comprising:
two or more medical diagnosis devices connected to the patient and providing diagnostic data.
an integrated diagnosis system that receives the diagnostic data, the integrated diagnosis system determines a diagnosis and provides a treatment for to respond to the diagnosis; and
two or more medical treatment devices connected to the patient that administer the treatment.
